(19) United States
(12) Patent Application Publication
(10) Pub. No.: US 2020/0071667 A1
(43) Pub. Date: Mar. 5, 2020

(54) COMPOSITION COMPRISING GLYCYRRHIZIN FOR INDUCING DIFFERENTIATION INTO MYELOID-DERIVED SUPPRESSOR CELL FROM MYELOID CELL

(71) Applicant: Konkuk University Glocal Industry-Academic Collabo, Chungcheongbuk-do (KR)

(72) Inventors: Seong Hyop Kim, Seoul (KR); Seung Hyun Lee, Seoul (KR); Eun Hye Seo, Incheon (KR)

(21) Appl. No.: 16/557,912

(22) Filed: Aug. 30, 2019

(30) Foreign Application Priority Data
Aug. 31, 2018 (KR) 10-2018-0103896

(51) Int. Cl. C12N 5/078

(52) U.S. Cl. CPC C12N 5/0634; C12N 2501/90; C12N 2501/999; C12N 2506/11

(57) ABSTRACT

Provided herein are a composition and method for inducing differentiation into myeloid-derived suppressor cells from myeloid cells using glycyrrhizin, and an immunosuppressive composition including the composition or myeloid-derived suppressor cells induced by the method.

Provided herein are also a composition and method for inducing differentiation into CD11b+Gr1 myeloid cells using glycyrrhizin, and a composition for proliferating myeloid cells which includes glycyrrhizin.

Thus, glycyrrhizin of the present disclosure has an effect of inducing differentiation into CD11b+Gr1 myeloid cells or myeloid-derived suppressor cells from myeloid cells in vivo and in vitro, and thus is effective as a composition for inducing differentiation into CD11b+Gr1 myeloid cells or myeloid-derived suppressor cells, which includes the glycyrrhizin.

In addition, when myeloid cells are treated with lipopolysaccharide (LPS) before being treated with the glycyrrhizin of the present disclosure in vitro, the effect of inducing differentiation into CD11b+Gr1 myeloid cells or myeloid-derived suppressor cells from myeloid cells is further enhanced.

### CROSS-REFERENCE TO RELATED APPLICATION

This application claims priority to and the benefit of Korean Patent Application No. 10-2018-0103896, filed on Aug. 31, 2018, the disclosure of which is incorporated herein by reference in its entirety.

### STATEMENT REGARDING FEDERALLY SPONSORED RESEARCH

This research has been supported by a grant of the Medical Research Center through the National Research Foundation of Korea, NRF, funded by the Ministry of Science, ICT and Future Planning, Republic Korea (grant number: NRF-2016R1A5A2012284).

### BACKGROUND

### 1. Field

The present disclosure relates to a composition and method for inducing differentiation into myeloid-derived suppressor cells from myeloid cells using glycyrrhizin, and an immunosuppressive composition comprising the composition or myeloid-derived suppressor cells induced by the method.

In addition, the present disclosure relates to a composition and method for inducing differentiation into CD11b+Gr1 myeloid cells using glycyrrhizin, and a composition for proliferating myeloid cells which includes glycyrrhizin.

### 2. Discussion of Related Art

Glycyrrhizin (glycyrrhizic acid or glycyrrhizinic acid) is contained in the roots of licorice (Glycyrrhiza glabra) and is a major sweetening ingredient. Glycyrrhizin is metabolized in the form of 11β-hydroxysteroid dehydrogenase, which is involved in metabolism of corticosteroids, or glycyrrhetinic acid which inhibits other enzymes.

Glycyrrhizin has a structure similar to a saponin and is known to have immunomodulatory, anti-inflammatory, hepatoprotective, neuroprotective, or antitumor activity. Specifically, glycyrrhizin regulates specific enzymes involved in inflammation or oxidative stress and down-regulates a specific pro-inflammatory regulator to thereby have an effect of protecting against damage due to infection or oxidative stress. Glycirizine is also capable of inhibiting the growth of sensitive tumors.

Myeloid-derived suppressor cells (MDSCs) are immune cells of the bone marrow stem cell line. MDSCs expand strongly in pathological situations such as chronic infections and cancer as a result of modified hematopoiesis. MDSCs are distinguished from other myeloid cell types having immune-stimulatory activity in that MDSCs have strong immunosuppressive activity, but are similar to other myeloid cell types in that MDSCs interact with other immune cells such as T cells, dendritic cells, macrophages, or natural killer cells to regulate the function thereof.

Korean Patent Publication No. 10-2017-0010731 relates to a method for inducing differentiation into myeloid-derived suppressor cells from cord blood CD34-positive cells and proliferating the same, and a use of the myeloid-derived suppressor cells, and discloses a composition and method for inducing differentiation into myeloid-derived suppressor cells by treating CD34+ cells isolated from human cord blood with GM-CSF and SCF.

Although there are disclosures of techniques for obtaining MDSCs in vitro, no studies or descriptions of the effect of glycyrrhizin capable of differentiation into CD11b+Gr1 myeloid cells, further into MDSCs from myeloid cells whether it be in vivo or in vitro have never been disclosed.

### SUMMARY

Therefore, the inventors of the present disclosure confirmed that, when myeloid cells were treated with glycyrrhizin, differentiation into myeloid cells having a phenotype of CD11b+Gr1+ is induced and furthermore, the effect of an increase in a ratio of myeloid-derived suppressor cells to myeloid cells or a ratio of myeloid-derived suppressor cells to CD11b+Gr1 myeloid cells was obtained, and thus the glycyrrhizin of the present disclosure could be effectively used for differentiation into myeloid-derived suppressor cells, and thus completed the present disclosure.

Therefore, one embodiment of the present disclosure provides a composition for inducing differentiation into myeloid-derived suppressor cells from myeloid cells, the composition including glycyrrhizin.

Another embodiment of the present disclosure provides an immunosuppressive composition including the above-described composition.

Another embodiment of the present disclosure provides a composition for inducing differentiation into CD11b+Gr1 myeloid cells from myeloid cells or a composition for proliferating myeloid cells, the composition including glycyrrhizin.

Another embodiment of the present disclosure provides a method of inducing differentiation into myeloid-derived suppressor cells or CD11b+Gr1 myeloid cells from myeloid cells, the method including:

i) isolating myeloid cells from a mammal;

ii) treating the myeloid cells isolated by process i) with lipopolysaccharide (LPS); and

iii) treating the LPS-treated myeloid cells with glycyrrhizin.

Another embodiment of the present disclosure provides an immunosuppressive composition including myeloid-derived suppressor cells differentiated using the above-described method.

Additional aspects will be set forth in part in the description which follows and, in part, will be apparent from the description, or may be learned by practice of the presented embodiments.

According to an aspect of an embodiment, there is provided a composition for inducing differentiation into myeloid-derived suppressor cells from myeloid cells, the composition including glycyrrhizin.

According to an aspect of another embodiment, there is provided an immunosuppressive composition including the composition.

According to an exemplary embodiment of the present disclosure, the immunosuppressive composition is used for preventing or treating organ transplant rejection, hematopoietic stem cell transplantation, an autoimmune disease, or an allergic disease, caused by immune hypersensitivity.

According to an aspect of another embodiment, there is provided a composition for inducing differentiation into CD11b+Gr1 myeloid cells from myeloid cells, the composition including glycyrrhizin.

According to an aspect of another embodiment, there is provided a composition for proliferating myeloid cells, the composition including glycyrrhizin.

According to an aspect of another embodiment, there is provided a method of inducing differentiation into myeloid-derived suppressor cells from myeloid cells, the method including:

i) isolating myeloid cells from a mammal;

ii) treating the isolated myeloid cells with lipopolysaccharide (LPS); and

iii) treating the LPS-treated myeloid cells with glycyrrhizin.

According to an exemplary embodiment of the present disclosure, a treatment concentration ratio of LPS of process ii) to glycyrrhizin of process iii) is in a range of 1:10 to 1:10⁵.

According to an aspect of another embodiment, there is provided an immunosuppressive composition including myeloid-derived suppressor cells differentiated according to the method.

According to an exemplary embodiment of the present disclosure, the immunosuppressive composition is used for preventing or treating organ transplant rejection, hematopoietic stem cell transplantation, an autoimmune disease, or an allergic disease, caused by immune hypersensitivity.

According to an aspect of another embodiment, there is provided a method of inducing differentiation into CD11b+Gr1 myeloid cells from myeloid cells, the method including:

i) isolating myeloid cells from a mammal;

ii) treating the isolated myeloid cells with lipopolysaccharide (LPS); and

iii) treating the LPS-treated myeloid cells with glycyrrhizin.

According to an exemplary embodiment of the present disclosure, a treatment concentration ratio of LPS of process ii) to glycyrrhizin of process iii) is in a range of 1:10 to 1:10⁵.

### BRIEF DESCRIPTION OF THE DRAWINGS

The above and other objects, features and advantages of the present disclosure will become more apparent to those of ordinary skill in the art by describing in detail illustrative embodiments with reference to the accompanying drawings, in which:

FIG. 1 illustrates a ratio of myeloid-derived suppressor cells (MDSCs) to myeloid cells, wherein the cells were obtained from blood, the heart, and the lungs of mice to which PBS (control), lipopolysaccharide (LPS) alone, or a combination of LPS and glycyrrhizin (GR) (LPS+GR) was administered, and it was confirmed that the ratio of MDSCs to myeloid cells was reduced in vivo in the case of LPS compared to a control regardless of the origin of myeloid cells, and the ratio of MDSCs to myeloid cells was significantly increased in the case of LPS+GR;

FIG. 2 illustrates histopathological analysis results and injury scores of heart tissues (A) or lung tissues (B) of mice to which PBS (control), LPS alone, or LPS+GR was administered, wherein the heart or lung tissues exhibited the infiltration of inflammatory cells, extensive alveolar wall thickening, alveolar hemorrhage, or the like in the LPS group and the LPS+GR group compared to the control, from which tissue injuries were identified, and for injury scores, in the case of the heart tissues, the injury scores of the LPS group and the LPS+GR group were significantly higher than that of control, and the injury scores of LPS and LPS+GR were similar to each other, and in the case of the lung tissues, the injury scores of the LPS group and the LPS+GR group were higher than that of control, and the injury score of the LPS+GR group was significantly lower than that of the LPS group;

FIG. 3 illustrates immunochemical analysis results and toll-like receptor 4 (TLR4) expression levels of heart tissues (A) or lung tissues (B) of mice to which PBS (control), LPS alone, or LPS+GR was administered, wherein it was confirmed that, in the case of both the heart and the lungs, the TLR4 expression levels of the LPS group and the LPS+GR group were higher than that of control, and the TLR4 expression level of the LPS+GR group was significantly lower than that of the LPS group;

FIG. 4 illustrates western blotting analysis results showing the TLR4 expression levels of heart tissues or lung tissues of mice to which PBS (control), LPS alone, or LPS+GR was administered, wherein it was confirmed that, similar to the immunochemical analysis results, in the case of both the heart and the lungs, the TLR4 expression levels of the LPS group and the LPS+GR group were higher than that of control, and the TLR4 expression level of the LPS+GR group was significantly lower than that of the LPS group;

FIG. 5 illustrates the results showing an increase in proliferation of myeloid cells in a manner dependent upon the concentration of glycyrrhizin when myeloid cells of mice were treated with various concentrations of glycyrrhizin in vitro, wherein the case of treatment of the myeloid cells with 300 μg/ml of glycyrrhizin exhibited the greatest proliferation rate of myeloid cells;

FIG. 6 illustrates the results showing an increase in the level of differentiation into CD11b+Gr1 myeloid cells in a manner dependent upon the concentration of glycyrrhizin when myeloid cells of mice were treated with various concentrations of glycyrrhizin in vitro, wherein differentiation into CD11b+Gr1 myeloid cells was most significantly increased upon treatment with 300 μg/ml of glycyrrhizin;

FIG. 7 illustrates an increase in the generation of inducible nitric oxide synthase (iNOS) in a manner dependent upon the concentration of glycyrrhizin when myeloid cells of mice were treated with various concentrations of glycyrrhizin in vitro; and

FIG. 8 illustrates the CFSE analysis results of measuring the activity of T cells in vitro using T cells as responder cells and myeloid cells of mice cultured for 48 hours after being treated with glycyrrhizin, as stimulator cells, from which it was confirmed that the activity of the T cells decreased as the content of the stimulator cells increased (responder cells: stimulator cells).

### DETAILED DESCRIPTION OF EXEMPLARY EMBODIMENTS

Exemplary embodiments of the present disclosure will be described in detail below with reference to the accompanying drawings. While the present disclosure is shown and described in connection with exemplary embodiments thereof, it will be apparent to those skilled in the art that various modifications can be made without departing from the spirit and scope of the present disclosure.

Hereinafter, the present disclosure will be described in detail.

As described above, there are various techniques for inducing differentiation into myeloid-derived suppressor cells (MDSCs) as the related art, but the relationship between myeloid cells and glycyrrhizin has never been studied.

Glycyrrhizin of the present disclosure has an effect of inducing differentiation into CD11b+Gr1 myeloid cells, further into MDSCs from myeloid cells in vivo or in vitro, and thus is effective as a composition for inducing differentiation into MDSCs.

Ammonium glycyrrhizinate of the present disclosure refers to glycyrrhizin (GR).

The term “differentiation” as used herein refers to specialization of the structure or function of cells during cell division and proliferation.

The term “immunosuppressive” or “inhibiting an immune response” as used herein refers to alleviation (relief of symptoms), treatment, or prevention of immune disorders due to hyperimmune or abnormal immune responses, or inhibition or delay of the onset thereof.

Therefore, the present disclosure provides a composition for inducing differentiation into MDSCs from myeloid cells, which includes glycyrrhizin.

Glycyrrhizin is a component contained in the roots of licorice (Glycyrrhiza glabra), and glycyrrhizin directly isolated from licorice or a commercially available product containing glycyrrhizin alone may be used.

The MDSCs inhibit the formation of T cell receptors through depletion of amino acids needed by lymphocytes or generate oxidative stress by producing inducible nitric oxide synthase (iNOS), reactive oxygen species (ROS), reactive nitrogen species (RNS), or the like, thereby inhibiting the proliferation or activity of T cells.

As a result of measuring the amount of generated iNOS or the activity of T cells after myeloid cells were treated with the above-described composition, it was confirmed that iNOS was significantly generated and the activity of T cells was significantly reduced, and thus the number of MDSCs was significantly increased compared to before the treatment (see Example 7). In addition, the effect of an increase in differentiation into MDSCs caused by glycyrrhizin is exhibited whether it be in vivo or in vitro.

Accordingly, the composition for inducing differentiation into MDSCs which includes glycyrrhizin of the present disclosure has an effect of inducing myeloid cells to be differentiated into MDSCs, and thus may increase the ratio of MDSCs to myeloid cells or the ratio of MDSCs to CD11b+Gr1 myeloid cells.

In the case of sepsis induced by LPS, it can be seen that a sepsis treatment effect due to glycyrrhizin administration is clearer than the case of sepsis induced by a specific pathogen. This is because the sepsis treatment effect may be affected by the interaction between a specific pathogen and glycyrrhizin. In addition, it takes about 12 hours or longer for myeloid cells in bone marrow to be differentiated and expressed in a specific organ, particularly about 18 hours or longer for myeloid cells to be differentiated and expressed in the heart or the lungs. Thus, to confirm myeloid cells differentiated and expressed in the heart or the lungs, it is required for mice to survive for a minimum of 18 hours or longer, and for this, LPS needs to be administered at a dose of 0.5 mg/kg or less.

When glycyrrhizin (GR) was administered to mice with sepsis induced by LPS, lung tissue injury caused by sepsis was significantly alleviated compared to heart tissue injury caused by sepsis. In addition, the TLR 4 expression level or the secretion amount of cytokines, which had been increased by LPS administration, was significantly reduced due to GR administration. Consequently, glycyrrhizin alleviates lung tissue injury caused by LPS-induced sepsis and inhibits TLR4 expression and the secretion of cytokines, thereby providing an effect of preventing or treating sepsis.

The present disclosure also provides an immunosuppressive composition including the above-described composition.

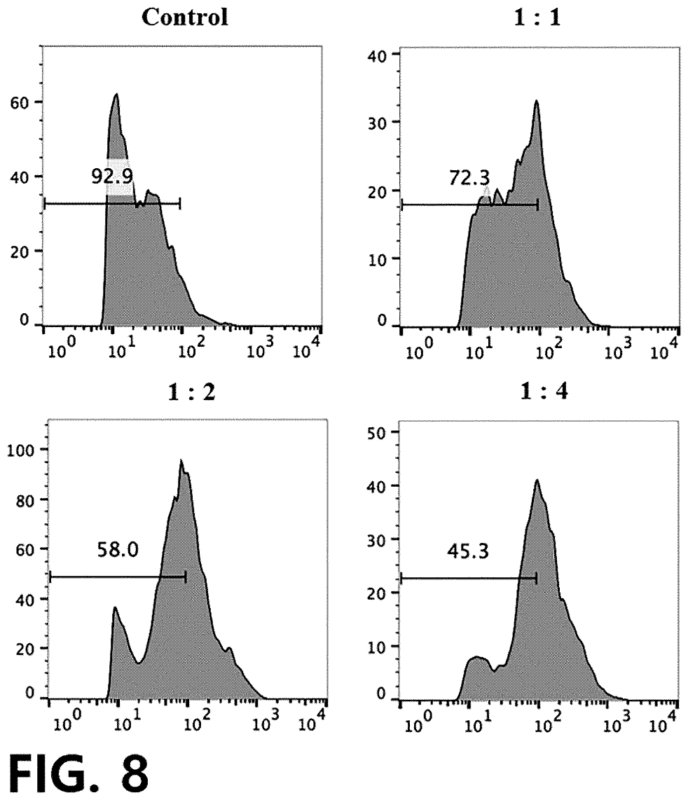

When differentiation into MDSCs from myeloid cells is induced using the composition of the present disclosure, the activity of T cells is significantly reduced by MDSCs having an immunosuppressive effect (see Example 7-2 and FIG. 8), and thus the composition of the present disclosure is effective as an immunosuppressive composition.

The immunosuppressive composition of the present disclosure may be in the form of various oral or parenteral formulations. When the composition is formulated, one or more buffers (e.g., saline or PBS), antioxidants, bacteriostatic agents, chelating agents (e.g., EDTA or glutathione), fillers, extenders, binders, adjuvants (e.g., aluminum hydroxide), suspending agents, thickening agents, wetting agents, disintegrating agents or surfactants, diluents, or excipients may be used.

Examples of solid preparations for oral administration include tablets, pills, powders, granules, capsules, and the like, and these solid preparations are formulated by mixing one or more compounds with at least one excipient, for example, starch (including corn starch, wheat starch, rice starch, potato starch, and the like), calcium carbonate, sucrose, lactose, dextrose, sorbitol, mannitol, xylitol, erythritol, maltitol, cellulose, methyl cellulose, sodium carboxymethylcellulose and hydroxypropylmethyl-cellulose, gelatin, or the like. For example, tablets or sugar tablets may be obtained by mixing an active ingredient with a solid excipient, pulverizing the mixture, adding a suitable adjuvant thereto, and then formulating the resultant mixture into a granular mixture.

In addition to simple excipients, lubricants such as magnesium stearate, talc, and the like are also used. Examples of liquid preparations for oral administration include suspensions, liquids for internal use, emulsions, syrups, and the like, and these liquid preparations may include, in addition to simple commonly used diluents, such as water and liquid paraffin, various types of excipients, for example, a wetting agent, a sweetener, a flavoring agent, a preservative, and the like. In addition, in some embodiments, crosslinked polyvinylpyrrolidone, agar, alginic acid, sodium alginate, or the like may be added as a disintegrating agent, and these preparations may further include an anticoagulant, a lubricant, a wetting agent, a flavoring, an emulsifier, a preservative, and the like.

Preparations for parenteral administration include an aqueous sterile solution, a non-aqueous solvent, a suspension, an emulsion, a freeze-dried preparation, a suppository, or the like. Non-limiting examples of the non-aqueous solvent and the suspension solvent include propylene glycol, polyethylene glycol, a vegetable oil such as olive oil, and an injectable ester such as ethyl oleate. Examples of suppository bases include Witepsol, Macrogol, Tween 61, cacao butter, laurin, glycerol, gelatin, and the like.

The composition of the present disclosure may be administered orally or parenterally, and for parenteral administration, the composition of the present disclosure may be formulated using a method known in the art into the form of: a dermatologic agent; an injection for intraperitoneal injection, intrarectal injection, intravenous injection, intramuscular injection, subcutaneous injection, intrauterine dural injection, or intracerebrovascular injection; a transdermal delivery agent; or a nasal inhalant.

Such injections must be sterile and protected from contamination of microorganisms such as bacteria and fungi. Non-limiting examples of suitable carriers for injections include solvents or dispersion media including water, ethanol, polyols (e.g., glycerol, propylene glycol, liquid polyethylene glycol, and the like), mixtures thereof and/or vegetable oils. For example, suitable carriers include isotonic solutions such as Hanks' solution, Ringer's solution, phosphate buffered saline (PBS) containing triethanol amine or sterile water for injection, 10% ethanol, 40% propylene glycol, and 5% dextrose, and the like. To protect the injections from microbial contamination, various antibacterial and antifungal agents such as parabens, chlorobutanol, phenol, sorbic acid, thimerosal, and the like may be further included. In addition, most of the injections may further include an isotonic agent such as sugar or sodium chloride.

Examples of the transdermal delivery agent include ointments, creams, lotions, gels, liquid for external application, pastes, liniments, aerosols, and the like. The term “transdermal delivery” as used herein means that the pharmaceutical composition is topically administered to the skin such that an effective amount of the active ingredient included in the pharmaceutical composition is delivered to the skin. For the inhalants, the compound used according to the present disclosure may be conveniently delivered in the form of aerosol spray from a pressurized pack or a nebulizer using a suitable propellant, for example, dichlorofluoromethane, trichlorofluoromethane, dichlorotetrafluoroethane, carbon dioxide, or other suitable gases. In the case of pressurized aerosols, a dosage unit may be determined by providing a valve configured to deliver a metered amount. For example, gelatin capsules and cartridges for use in inhalers or blowers may be formulated to include a powder mixture of a compound and a suitable powder base such as lactose or starch. Formulations for parenteral administration are described in the reference, which is a prescription commonly known for all pharmaceutical chemistry (Remington's Pharmaceutical Science, 15th Edition, 1975. Mack Publishing Company, Easton, Pa. 18042, Chapter 87: Blaug, Seymour).

The composition of the present disclosure is administered in a pharmaceutically effective amount. The term “pharmaceutically effective amount” as used herein refers to an amount sufficient to treat diseases at a reasonable benefit/risk ratio applicable to medical treatment, and an effective dosage level may be determined according to factors including type of diseases of patients, the severity of disease, the activity of drugs, sensitivity to drugs, administration time, administration route, excretion rate, treatment periods, and simultaneously used drugs, and other factors well known in the medical field. The composition of the present disclosure may be administered as an individual therapeutic agent or in combination with other therapeutic agents, may be administered sequentially or simultaneously with existing therapeutic agents, and may be administered in a single dose or multiple doses. That is, a total effective amount of the composition of the present disclosure may be administered to a patient in a single dose, and may be administered by a fractionated treatment protocol which is administered in multiple doses for a long period of time. It is important to administer the composition in the minimum amount that enables achievement of the maximum effects without side effects in consideration of all the above-described factors, and this may be easily determined by those of ordinary skill in the art.

A dosage of the immunosuppressive composition of the present disclosure varies depending on the body weight, age, and gender of a patient, health conditions, diet, administration time, administration method, excretion rate, and the severity of disease. A daily dosage thereof may be administered parenterally in an amount of about 0.01 mg to about 50 mg, for example, about 0.1 mg to about 30 mg per body weight (1 kg) based on the glycyrrhizin, and a daily dosage thereof may be administered orally in a single dose or multiple doses in an amount of about 0.01 mg to about 100 mg, for example, about 0.01 mg to about 10 mg per body weight (1 kg), based on the glycyrrhizin of the present disclosure. However, the dosage may be increased or decreased according to administration route, the severity of obesity, gender, body weight, age, and the like, and thus the dosage is not intended to limit the scope of the present disclosure in any way.

The composition of the present disclosure may be used alone or in combination with surgery, radiation therapy, hormone therapy, chemotherapy, and methods using a biological response modifier.

The immunosuppressive composition of the present disclosure may also be provided in the form of a formulation for external use, including glycyrrhizin as an active ingredient. When used as a preparation for external application to the skin, the composition may further include adjuvants commonly used in dermatology, such as other ingredients commonly used in preparations for external application to the skin, for example, fatty substances, organic solvents, solubilizing agents, thickeners and gelling agents, softeners, antioxidants, suspending agents, stabilizers, foaming agents, fragrances, surfactants, water, ionic or non-ionic emulsifiers, fillers, metal ion blocking agents, chelating agents, preservatives, vitamins, blocking agents, wetting agents, essential oils, dyes, pigments, hydrophilic or lipophilic active agents, lipid vesicles, or the like. In addition, the above-listed ingredients may be introduced in an amount generally used in the dermatology field.

When the immunosuppressive composition of the present disclosure is provided as a dermatologic agent, the preparation may be in the form of a formulation such as ointment, a patch, gel, a cream, an aerosol, or the like, but the present disclosure is not limited thereto.

The immunosuppressive composition of the present disclosure may be used for the prevention or treatment of organ transplant rejection, hematopoietic stem cell transplantation, an autoimmune disease or an allergic disease caused by immune hypersensitivity, but the present disclosure is not limited thereto.

The present disclosure also provides a composition for inducing differentiation into CD11b+Gr1 myeloid cells from myeloid cells, which includes glycyrrhizin.

The glycyrrhizin is the same as that used in the composition for inducing differentiation into MDSCs, and thus the above description can be referred to.

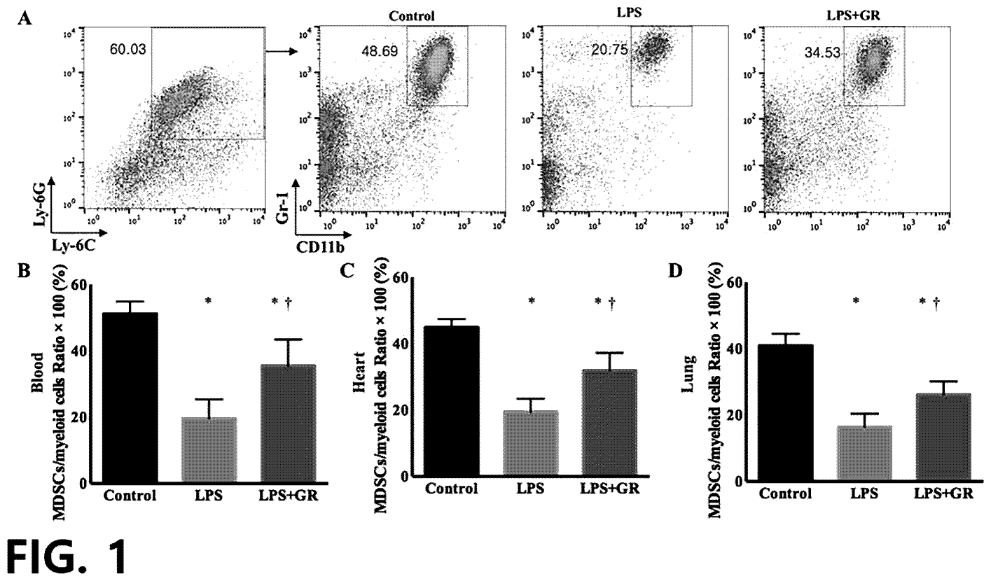

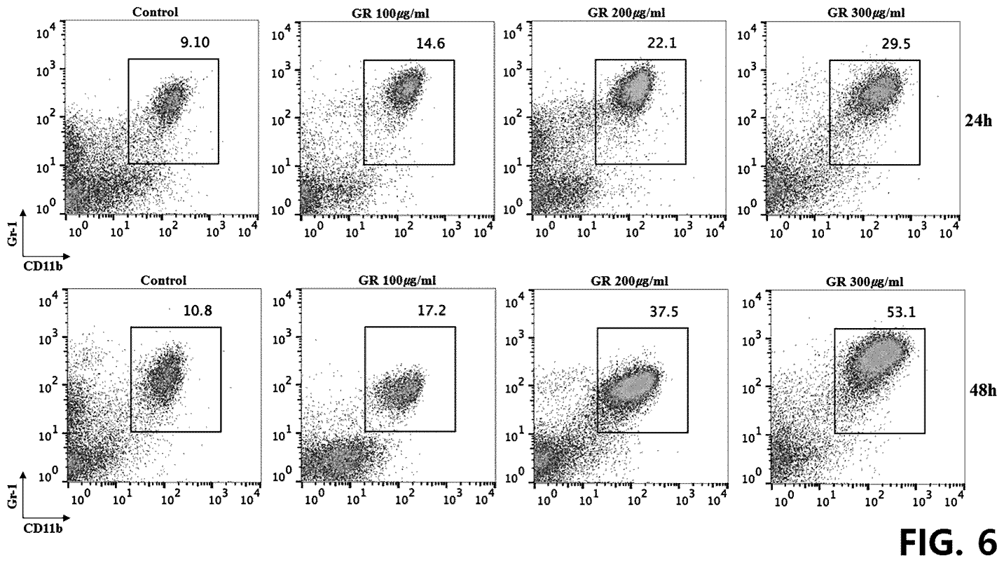

Glycyrrhizin induces differentiation into CD11b+Gr1 myeloid cells from myeloid cells, and the differentiation induction effect is increased in a manner dependent upon the concentration of glycyrrhizin (see FIGS. 1 and 6), and thus is effectively used as a composition for inducing differentiation into CD11b+Gr1 myeloid cells. Myeloid cells having a phenotype of CD11b+Gr1+, i.e., myeloid cells having a cluster of differentiation (CD) of CD11b and Gr1, also have a CD of CD31 (see Example <6-1>).

The present disclosure also provides a composition for proliferating myeloid cells, which includes glycyrrhizin.

The glycyrrhizin is the same as that used in the composition for inducing differentiation into MDSCs, and thus the above description can be referred to.

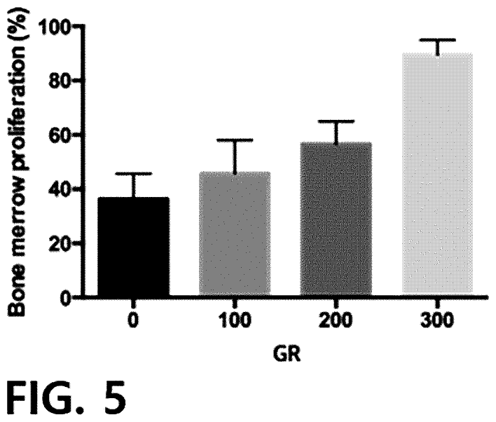

When myeloid cells of mice are treated with the glycyrrhizin, the proliferation of the myeloid cells increases, and the proliferation rate is increased in a concentration-dependent manner (see Example 6 and FIG. 5), and thus the glycyrrhizin is effective as a composition for proliferating myeloid cells.

The present disclosure also provides a method of inducing differentiation into MDSCs or CD11b+Gr1 myeloid cells from myeloid cells, the method comprising:

i) isolating myeloid cells from a mammal;

ii) treating the myeloid cells isolated by process i) with lipopolysaccharide (LPS); and

iii) treating the LPS-treated myeloid cells with glycyrrhizin.

The mammal refers to all animals classified as mammals, such as humans, domestic animals, farm livestock, pet animals, or the like.

The method for inducing differentiation of myeloid cells of the present disclosure may be used in vitro, which is because when LPS is administered in vivo, differentiation into or the proportion of MDSCs is reduced (see Example 2 and FIG. 1), and thus the effect of inducing differentiation into MDSCs due to glycyrrhizin administration is insignificant.

Specifically, process ii) is a process of stimulating myeloid cells with LPS to induce differentiation thereof, and the LPS is a cell wall component of Gram-negative bacteria and LPS directly isolated from Gram-negative bacteria or a commercially available product including LPS alone may be used.

Process iii) is a process of inducing differentiation into MDSCs or CD11b+Gr1 myeloid cells by treating the myeloid cells stimulated to be differentiated with glycyrrhizin.

A treatment concentration ratio of LPS to glycyrrhizin (LPS:glycyrrhizin) may range from about 1:10 to about 1:10⁵, for example, about 1:10² to 10:10⁴, for example, 1:10³.

The present disclosure also provides an immunosuppressive composition including MDSCs differentiated using the above-described method.

For the description of the immunosuppressive composition, refer to the description of the immunosuppressive composition including the above-described composition.

The immunosuppressive composition may be used for the prevention or treatment of organ transplant rejection, hematopoietic stem cell transplantation, an autoimmune disease, or an allergic disease, which is caused by immune hypersensitivity, but the present disclosure is not limited thereto.

Hereinafter, the present disclosure will be described in further detail with reference to the following examples. It will be obvious to those of ordinary skill in the art that these examples are provided for illustrative purposes only and are not intended to limit the scope of the present disclosure.

### Example 1

Experiment Preparation

Animal experiments were performed according to the National Institutes of Health (NIH) guidelines for the care and use of experimental animals. This study was conducted with the approval of the Konkuk University Animal Care and Use Committee (KU16062). 8-week-old male C57BL/6 mice without specific pathogens were used, and as needed, experimental groups were divided into three groups: a control, an LPS group, and an LPS+GR group.

Specifically, 0.1 ml of phosphate buffered saline (PBS; Gibco, Grand Island, N.Y., USA) was injected intraperitoneally (i.p.) into mice of the control, and 12 hours after the injection, 0.1 ml of PBS was intraperitoneally injected again into each mouse. 1 μg of LPS (Escherichia coli serotype 0111:B4, Sigma-Aldrich, St. Louis, Mo., USA) dissolved in 0.1 ml of PBS was injected intraperitoneally (i.p.) into mice of the LPS group to induce sepsis, and 12 hours after sepsis induction, 0.1 ml of PBS was intraperitoneally injected into each mouse. 1 μg of LPS and 100 mg/kg of ammonium glycyrrhizinate (GR; Abcam Inc., Cambridge, UK) dissolved in 0.1 ml of PBS were injected intraperitoneally (i.p.) into mice of the LPS+GR group, and 12 hours after the injection, 100 mg/kg of ammonium glycyrrhizinate dissolved in 0.1 ml of PBS was intraperitoneally injected into each mouse.

### Example 2

Effect of Glycyrrhizin on Inducing Differentiation of Myeloid Cells

<2-1> Case in which Glycyrrhizin was Administered to Mice with Sepsis (In Vivo)

To confirm whether treatment of mice with sepsis induced by LPS with glycyrrhizin (GR) affects the differentiation direction of myeloid cells, flow cytometric analysis was used, and myeloid cells obtained from blood, the heart, and the lungs were used.

Specifically, myeloid cells were obtained from mice of each of the three groups prepared in Example 1 above. Blood was obtained from the abdominal aorta of mice with a 2 ml syringe coated with heparin and collected in a tube previously coated with ethylenediaminetetraacetic acid. After centrifugation at 1,500 rpm for 5 minutes at 25° C., the supernatant was separated to obtain pellets. Myeloid cells were obtained from the pellets through density gradient centrifugation using a Biocoll gradient solution (Biochrom GmbH, Berlin, Germany). Heart and lung tissues were cut to a size of 1 mm³ on ice and washed. The tissues were then digested with 1 mg/ml of collagenase type 1 (Sigma-Aldrich) in PBS (5 ml) at 37° C. for 60 minutes. After incubation, mononuclear cells of the digested solution were filtered through a 70-μm cell filter (SPL Life Science, Pocheon, Korea).

The myeloid cells obtained from blood, the heart, or the lungs were washed with fluorescence-activated cell sorting (FACS) buffer (PBS containing 1% bovine serum albumin and 0.01% NaN₃). After washing, Gr-1-allophycocyanin (Gr-1-APC; eBioscience, San Diego, Calif., USA), F4/80-fluorescein isothiocyanate (FITC; BioLegend, San Diego, Calif., USA), leukocyte antigen-6G-phycoerythrin (Ly-6G-PE; Miltenyi Biotec, Bergisch Gladbach, Germany), Ly-6C-peridinin chlorophyll protein (Ly-6C-PerCP; Miltenyi Biotec, Bergisch Gladbach, Germany), and CD11b-APC/cy7 (BioLegend) antibodies were used to distinguish CD11b+Gr1 myeloid cells. The corresponding isotype antibodies were used with all samples as a control. Flow cytometric analysis was performed using a FACSAria™ instrument (Becton Dickinson, Franklin Lakes, N.J., USA).

As a result, as illustrated in FIG. 1, for the ratio of myeloid-derived suppressor cells (MDSCs) to the myeloid cells obtained from blood, the heart, or the lungs of mice of each experimental group, the ratio of MDSCs to myeloid cells (MDSCs/myeloid cells) of each of the LPS group and the LPS+GR group was significantly reduced compared to the control. However, the ratio of MDSCs to myeloid cells of the LPS+GR group was significantly increased compared to the LPS group. Thus, it was confirmed that differentiation into MDSCs was induced when mice with sepsis induced by LPS were treated with glycyrrhizin (GR).

<2-2> Case where Myeloid Cells of Sepsis Mice were Treated with Glycyrrhizin (In Vitro)

To verify the in vivo experimental results of Example <2-1>, flow cytometric analysis was used to confirm whether differentiation into CD11b+Gr1 myeloid cells or MDSCs from myeloid cells was induced when myeloid cells of mice were treated with glycyrrhizin (GR).

Specifically, myeloid cells were obtained by washing the femoral region of each of 8-week-old male C57BL/6 male mice without specific pathogens with a Roswell Park Memorial Institute (RPMI) 1640 medium in a sterile state using a syringe with a 27-gauge needle. Red blood cells (RBCs) of the collected bone marrow were lysed with an erythrolysis solution, and then the remaining cells were treated with 10% fetal bovine serum, 40 ng/ml of IL-6, and 40 ng/ml of granulocyte-macrophage colony stimulating factor at 37° C. Thereafter, only 1 ng/ml of LPS was treated (LPS group) or 100 μg/ml of GR was additionally treated (LPS+GR group) after LPS treatment. The control was treated with 0.1 ml of PBS. After incubation for 3 days, the same flow cytometric analysis as in Example <2-1> was performed to measure the absolute number of myeloid cells of each group, and then all the obtained data was analyzed using FlowJo software (Tree Star Inc., Ashland, Oreg., USA) to determine the ratio of MDSCs to CD11b+Gr1 myeloid cells.

As a result, as shown in Table 1 below, the numbers of MDSCs and CD11b+Gr1 myeloid cells of the LPS+GR group were significantly increased compared to the control and LPS group, and the ratio of MDCSs to CD11b+Gr1 myeloid cells was also significantly increased compared to the control and the LPS group.

TABLE 1

| | Control | LPS | LPS + GR |
|---|---|---|---|
| Total cell count | 50,000 | 47,300 ± 1,200 | 48,700 ± 800 |
| MDSCs | 4,620 ± 60 | 5,200 ± 300 | 10,800 ± 700 |
| | (9.24 ± 0.12) | (11.23 ± 1.25) | (21.42 ± 3.10) |
| CD11b + Gr1 | 11,700 ± 100 | 16,500 ± 400 | 17,700 ± 200 |
| myeloid cells | (23.40 ± 0.03) | (34.61 ± 4.51) | (34.77 ± 8.00) |
| MDSCs/CD11b + | 40.00 ± 1.45 | 32.45 ± 2.69 | 60.40 ± 3.17 |
| Gr1 myeloid cells × | | | |
| 100 (%) | | | |

### Example 3

Confirmation of Whether Tissue Injury was Alleviated by Glycyrrhizin

Histopathologic analysis was used to determine whether heart or lung tissue injury caused by sepsis is alleviated when mice with sepsis induced by LPS were treated with glycyrrhizin.

Specifically, the heart tissues and lung tissues of mice of each experimental group prepared in Example 1 were fixed in a 4% paraformaldehyde solution (Biosesang, Seongnam, Korea) at 25° C. overnight and inserted into paraffin blocks. The tissues were then cut to a 4 μm thickness using a microtome and stained with hematoxylin (Vector Laboratories, Burlingame, Calif., USA) and eosin (Sigma-Aldrich) and then examined using an optical microscope. Heart (including left and right ventricles) or lung injury was scored based on Table 2 below. Each score was determined by a 5-point scale such as 0: minimal injury, 1: minor injury, 2: moderate injury, 3: severe injury, and 4: maximum injury.

TABLE 2

| | 0 point | 1 points | 2 points | 3 points | 4 points |
|---|---|---|---|---|---|
| Heart | No | Isolated | Intensive | Intensive | At least 50% |
| | injury | muscle | damage in | damage in two | distributed |
| | | cell | one area | or more areas | myocardium |
| | | damage | | | damage |
| Lungs | No | Alveolar | bleeding | Infiltration or | Hardening of |
| | injury | congestion | | aggregation of | alveolar wall |
| | | | | neutrophils | or hyaline |
| | | | | into airspace | membrane |
| | | | | or blood | structure |
| | | | | vessel walls | |

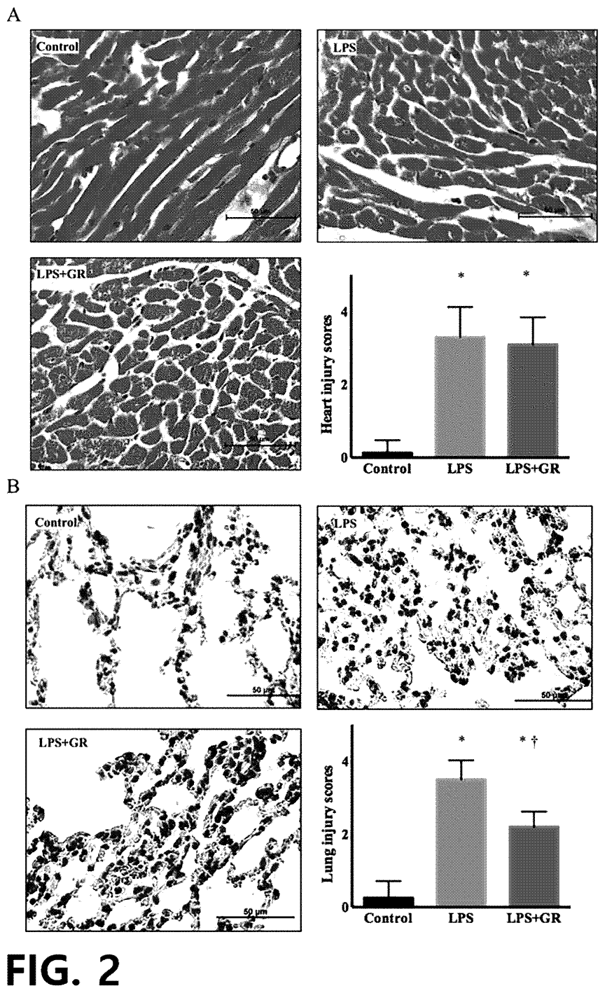

As a result, as illustrated in FIG. 2, the heart tissues or the lung tissues were normal in the control, whereas the LPS group and the LPS+GR group exhibited inflammatory cell infiltration, extensive alveolar wall thickening, alveolar bleeding, and the like. For the injury scores, the LPS group and the LPS+GR group showed similar injury scores in the heart tissues, which are significantly higher than that of the control (LPS group: 3.3±0.8, LPS+GR group: 3.1±0.7, control: 0.4, p<0.05). In the case of the lung tissues, the LPS group and the LPS+GR group showed significantly higher injury scores compared to that of the control, whereas the injury score of the LPS+GR group was significantly lower than that of the LPS group (LPS group: 3.5±0.5, LPS+GR group: 2.2±0.4, control: 0.3±0.5, p<0.05).

### Example 4

Confirmation of Whether Glycyrrhizin Inhibits Secretion of Cytokines

It was examined whether the secretion levels of inflammatory cytokines related to sepsis were inhibited when mice with sepsis induced by LPS were treated with glycyrrhizin. The secretion levels of inflammatory cytokines were confirmed by analyzing bronchoalveolar lavage fluid (BALF) and blood.

Specifically, for the analysis of bronchoalveolar lavage fluid and blood, 12 hours after the final injection into mice of each experimental group of Example 1, 1 μg/g of Zoletil®50 (a combined preparation of tiletamine and zolazepam, Virbac Laboratories, Carros, France) as an anesthetic and 0.1 U/g of heparin sodium (Green Cross Co., Yongin, Korea) were intraperitoneally injected into each mouse to minimize the effect of aches or pains on immunity. Each mouse was sacrificed by cervical dislocation and subjected to bronchoalveolar lavage (BAL). Lung washes were performed three times using 0.5 ml cold PBS, and BALF was obtained by intubation with a 19-gauge catheter. Blood was obtained by the method of Example <2-1>. The levels of tumor necrosis factor (TNF)-α, IL-6, and IL-1β in BALF and blood were measured using a mouse enzyme-linked ELISA kit (R&D Systems Inc., Minneapolis, Minn., USA) according to the manufacturer's instructions. Samples collected from each group were analyzed in triplicate.

As a result, as shown in Table 3 below, the secretion of all cytokines in BALF or blood was significantly increased in the LPS group and the LPS+GR group compared to the control. However, the secretion of all cytokines in BALF or blood was significantly decreased in the LPS+GR group compared to the LPS group. In particular, there was no significant difference in the secretion of IL-6 in blood between the control and the LPS+GR group.

TABLE 3

| | BALF | | | Blood | | |
|---|---|---|---|---|---|---|
| | Control | LPS | LPS + GR | Control | LPS | LPS + GR |
| TNF-α (pg/μl) | 70.11 ± 21.76 | 284.20 ± 56.96 | 167.81 ± 37.27 | 18.32 ± 6.64 | 98.78 ± 9.17 | 49.52 ± 13.20 |
| IL-6 (pg/μl) | 11.45 ± 3.56 | 650.26 ± 11.02 | 132.66 ± 13.44 | 36.51 ± 7.96 | 172.63 ± 14.22 | 38.16 ± 12.01 |
| IL-1β (pg/μl) | 15.52 ± 2.43 | 164.82 ± 17.81 | 34.38 ± 6.66 | 21.22 ± 4.23 | 126.28 ± 7.61 | 72.81 ± 11.02 |

### Example 5

Confirmation of Whether Glycyrrhizin Inhibits TLR4 Expression

It was determined whether TLR4 expression levels were inhibited when mice with sepsis induced by LPS were treated with glycyrrhizin. The expression level of Toll-like receptor 4 (TLR4) was confirmed using immunohistochemistry and western blot analysis.

Specifically, for immunohistochemical staining for TLR4, the heart or lung tissues of mice of each experimental group of Example 1 were cut to 4 μm after deparaffinization treatment. The sections were cultured in a blocking solution (Vector Laboratories) at a constant temperature for 1 hour and reacted with TLR4 rabbit polyclonal antibodies (Abcam Inc.) diluted at 1:100 and 4° C. After washing with PBS, the sections were incubated with diluted biotinylated secondary antibodies for 1 hour. After washing, each section was treated with an ABC reagent (Vector Laboratories) at 25° C. for 1 hour and allowed to bind thereto with a 3,30-diaminobenzidine reagent (Vector Laboratories). The slices were stained with hematoxylin as an antagonist and rehydrated, and covered-slipped using a mounting medium (Vector Laboratories). Images were captured using a microscope (Nikon, Tokyo, Japan). TLR4 intensity was quantified using NIH Image J software.

Western blotting was performed to confirm the intensity of TLR4 staining. Proteins were extracted from homogenized samples (50 μg/lane) and electrophoresed on Tris-glycine gels (Invitrogen, Carlsbad, Calif., USA) and transferred to polyvinylidene difluoride membranes. For immunoblotting, each membrane was cultured with anti-TLR4 (1:200, at 4° C. for 1 day, Santa Cruz Biotechnology, Santa Cruz, Calif., USA) antibodies, followed by incubation in 5% skimmed dry milk at 4° C. for 1 day. After washing three times with Tris-PBS, each membrane was incubated with peroxidase-binding secondary antibodies (1:5,000) for 1 hour at room temperature. The membrane was then developed using enhanced chemiluminescence (Amersham Pharmacia Biotech, Piscataway, N.J., USA). Each membrane was stripped and re-blotted against glycanaldehyde 3-phosphate dehydrogenase (1:2,000 at room temperature for 30 minutes, 1:20,000 for 30 minutes at room temperature (secondary), Biodesign International, Saco, Me., US) to confirm equivalent protein loading for each lane. Assays were repeated three times to ensure reproducibility. Band intensity was quantified using Luminescent Image Analyzer LAS-3000 (FUJIFILM Medical Systems, Hanover Park, Ill., USA). The density of each band was analyzed using NIH Image J software.

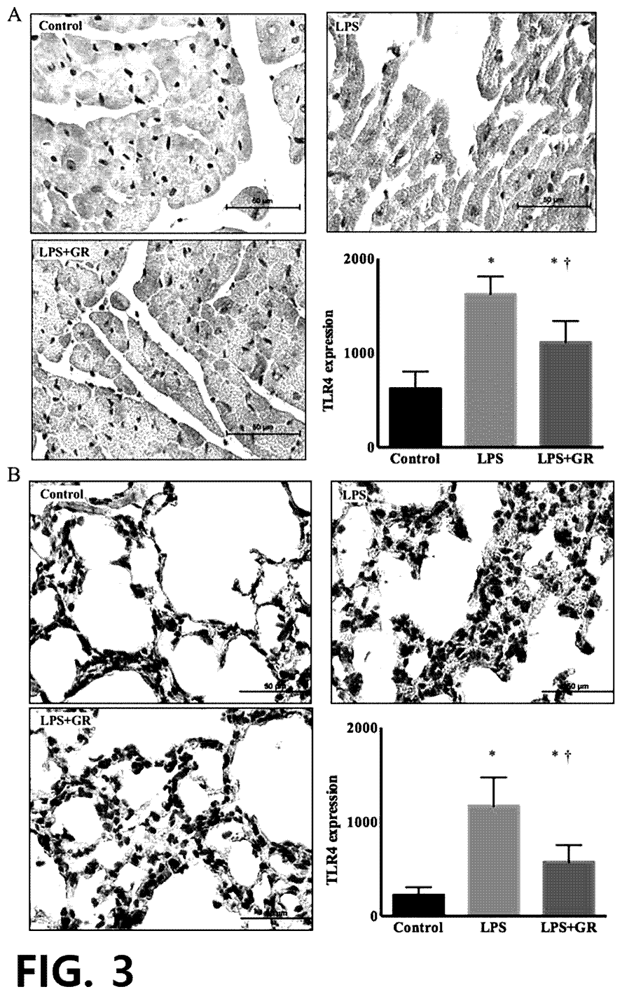

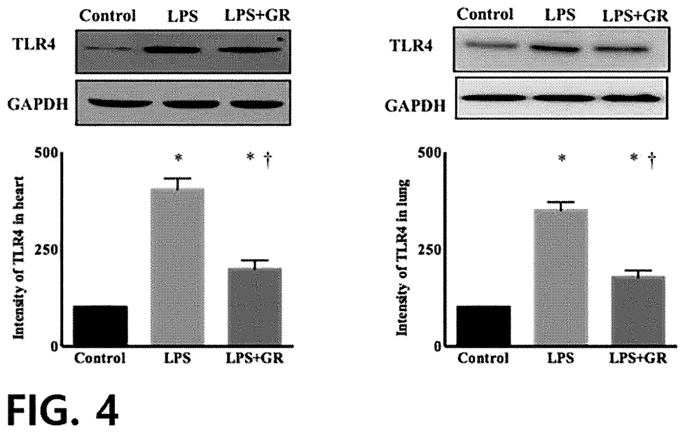

As a result, when the intensity of TLR4 was analyzed by immunohistochemistry, as illustrated in FIG. 3, the expression of TLR4 in the LPS group and the LPS+GR group was significantly higher in both the heart and the lungs compared to the control, whereas the expression level of TLR4 of the LPS+GR group was significantly lower than that of the LPS group. The western blotting analysis results of the TLR4 intensity were similar to the immunohistochemistry analysis results as illustrated in FIG. 4 (For the heart, LPS group: 403.9±29.3, LPS+GR group: 198.8±23.2, control: 101.4±1.8, p<0.05; for the lungs, LPS group: 350.7±21.3, LPS+GR group: 178.3±17.4, control: 101.0±1.7, p<0.05).

### Example 6

Effect of Glycyrrhizin on Inducing Differentiation into CD11b+Gr1 Myeloid Cells

<6-1> Confirmation of Myeloid Cell Proliferation Effect after Glycyrrhizin Treatment

It was examined whether myeloid cells are capable of proliferating even when treated with glycyrrhizin alone without stimulation of the myeloid cells with LPS.

Specifically, myeloid cells having a phenotype of CD31+ were isolated from mouse myeloid cells obtained in the same manner as in Example 2-2. The myeloid cells were treated with glycyrrhizin at a concentration of 100 μg/ml, 200 μg/ml, or 300 μg/ml, and then cultured for 48 hours. As a control, the myeloid cells were treated with 0.1 ml of PBS. Subsequently, the expression level of myeloid cells was examined by the same flow cytometric analysis as that used in Example <2-1>.

As a result, as illustrated in FIG. 5, it was confirmed that the proliferation of the myeloid cells was increased in a manner dependent upon the concentration of glycyrrhizin with which the myeloid cells were treated without stimulation by LPS.

<6-2> Confirmation of Treatment Concentration of Glycyrrhizin and Culture Time

Flow cytometric analysis was performed to determine whether the concentration of glycyrrhizin with which myeloid cells were treated or culture time affects differentiation into CD11b+Gr1 myeloid cells from myeloid cells.

Specifically, mouse myeloid cells were obtained in the same manner as in Example <2-2>, treated with glycyrrhizin at a concentration of 100 μg/ml, 200 μg/ml, or 300 μg/ml, and then cultured for 24 hours or 48 hours. As a control, the myeloid cells were treated with 0.1 ml of PBS. Subsequently, the expression level of the myeloid cells was examined by the same flow cytometric analysis as that used in Example <2-1>.

From the results, it was confirmed as illustrated in FIG. 6 that the expression level of CD11b+Gr1 myeloid cells was increased in a concentration-dependent manner when the myeloid cells were treated with glycyrrhizin and cultured for 24 hours, and the case of treatment with a high concentration (300 μg/ml) of glycyrrhizin exhibited a three or more-fold increase (29.5%) compared to the control (9.10%). It was also confirmed that the case of the high concentration (300 μg/ml) exhibited a five or more-fold increase (53.1%) compared to the control (10.8%) when the culture time was extended to 48 hours.

### Example 7

Confirmation of Whether Glycyrrhizin Induces Differentiation into MDSCs

<7-1> iNOS Measurement for Confirming Differentiation into MDSCs from CD11b+Gr1 Myeloid Cells

As confirmed in Example 6 showing an increase in differentiation amount of CD11b+Gr1 myeloid cells due to glycyrrhizin treatment, iNOS measurement was performed to confirm whether the amount of differentiation into MDSCs from CD11b+Gr1 myeloid cells was also increased.

Specifically, myeloid cells were isolated from mice of the control of Example 1, treated with glycyrrhizin at a concentration of 100 μg/ml, 200 μg/ml, or 300 μg/ml, and then cultured for 48 hours. Thereafter, RT-PCR was performed. As a negative control, the myeloid cells were treated with 0.1 ml of PBS, and GAPDH was used as an expression control.

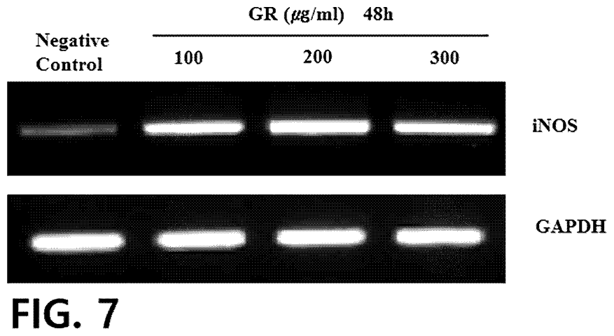

As a result, it was confirmed as illustrated in FIG. 7 that the expression level of iNOS was increased in a concentration-dependent manner when the myeloid cells were treated with glycyrrhizin compared to the negative control.

<7-2> T Cell Activity Measurement for Confirming Differentiation into MDSCs from CD11b+Gr1 Myeloid Cells

As confirmed in Example 6 showing an increase in differentiation amount of CD11b+Gr1 myeloid cells due to glycyrrhizin treatment, the activity of T cells was measured using a carboxyfluorescein succinimidyl ester (CFSE) analysis method to confirm whether the amount of differentiation into MDSCs from CD11b+Gr1 myeloid cells was also increased.

Specifically, mouse myeloid cells were obtained in the same manner as in Example <2-2>, treated with 300 μg/ml of glycyrrhizin, and then cultured for 48 hours. Subsequently, CD11b+Gr1 myeloid cells induced to be differentiated were stained and separated with FACSAria to be used as stimulator cells, and for responder cells, T cells were isolated with FACSAria from splenocytes of mice of the control of Example 1 and stained with CFSE. The responder cells and the stimulator cells were treated with CD3 and CD28, which induce the activity of T cells, at a ratio of 1:1, 1:2, or 1:4 and further cultured for 3 days.

As a result, it was confirmed as illustrated in FIG. 8 that the higher the proportion of the stimulator cells (CD11b+Gr1 myeloid cells), the lower the activity of T cells.

For statistical analysis, the main result was the ratio of MDSCs to CD11+GR1 myeloid cells. According to a preliminary study using 6 mice with LPS-induced sepsis, the blood ratio was 18.4±3.13%. Sample size was calculated with G-Power 3.1.9.2. The sample size of 10 mice per experimental group was calculated to determine a significant difference. Analysis was performed using the IBM SPSS Statistics 21.0 software package (IBM, Inc., Armonk, N.Y., USA) and GraphPad Prism 6.0 software (GraphPad Software, Inc., La Jolla, Calif., USA). Statistical significance between the three experimental groups was examined by one way analysis. Statistical significance between the two experimental groups was confirmed by a t-test. All data is expressed as the number of mice and mean±standard deviation. P values less than 0.05 were considered to indicate statistical significance.

As is apparent from the foregoing description, glycyrrhizin of the present disclosure has an effect of inducing differentiation into CD11b+Gr1 myeloid cells or myeloid-derived suppressor cells from myeloid cells in vivo and in vitro, and thus is effective as a composition for inducing differentiation into CD11b+Gr1 myeloid cells or myeloid-derived suppressor cells, which includes the glycyrrhizin.

In addition, when myeloid cells are first treated with lipopolysaccharide (LPS) before being treated with the glycyrrhizin of the present disclosure in vitro, the effect of inducing differentiation into CD11b+Gr1 myeloid cells or myeloid-derived suppressor cells from myeloid cells is further enhanced.

It will be apparent to those skilled in the art that various modifications can be made to the above-described exemplary embodiments of the present disclosure without departing from the spirit or scope of the present disclosure. Thus, it is intended that the present disclosure covers all such modifications provided they come within the scope of the appended claims and their equivalents.

1. A composition for proliferating myeloid cells or for inducing differentiation into myeloid-derived suppressor cells from myeloid cells, the composition comprising glycyrrhizin.

2. An immunosuppressive composition comprising the composition of claim 1.

3. The immunosuppressive composition of claim 2, wherein the immunosuppressive composition is used for preventing or treating organ transplant rejection, hematopoietic stem cell transplantation, an autoimmune disease, or an allergic disease, caused by immune hypersensitivity.

4. The composition of claim 1 wherein the ratio of myeloid-derived suppressor cells to myeloid cells or the ratio of myeloid-derived suppressor cells to CD11b+Gr1 myeloid cells is increased.

5. (canceled)

6. A method of inducing differentiation into myeloid-derived suppressor cells from myeloid cells, the method comprising:
i) isolating myeloid cells from a mammal;
ii) treating the isolated myeloid cells with lipopolysaccharide (LPS); and
iii) treating the LPS-treated myeloid cells with glycyrrhizin.

7. The method of claim 6, wherein a treatment concentration ratio of LPS of process ii) to glycyrrhizin of process iii) is in a range of 1:10 to 1:10⁵.

8. An immunosuppressive composition comprising myeloid-derived suppressor cells differentiated according to the method of claim 6.

9. The immunosuppressive composition of claim 8, wherein the immunosuppressive composition is used for preventing or treating organ transplant rejection, hematopoietic stem cell transplantation, an autoimmune disease, or an allergic disease, caused by immune hypersensitivity.

10. A method of inducing differentiation into CD11b+Gr1 myeloid cells from myeloid cells, the method comprising:
i) isolating myeloid cells from a mammal;
ii) treating the isolated myeloid cells with lipopolysaccharide (LPS); and
iii) treating the LPS-treated myeloid cells with glycyrrhizin.

11. The method of claim 10, wherein a treatment concentration ratio of LPS of process ii) to glycyrrhizin of process iii) is in a range of 1:10 to 1:10⁵.
